(19) United States
(12) Patent Application Publication
(10) Pub. No.: US 2021/0353351 A1
(43) Pub. Date: Nov. 18, 2021

(54) Thermal Devices and Methods of Visceral Fat Reduction

(71) Applicants: Meital MAZOR, San Diego, CA (US); Alexei BABKIN, Dana Point, CA (US); Rafi MAZOR, San Diego, CA (US)

(72) Inventors: Meital MAZOR, San Diego, CA (US); Alexei BABKIN, Dana Point, CA (US); Rafi MAZOR, San Diego, CA (US)

(21) Appl. No.: 17/277,409

(22) PCT Filed: Sep. 18, 2019
(86) PCT No.: PCT/US2019/051746

§ 371 Date: Mar. 18, 2021

Related U.S. Application Data
(60) Provisional application No. 62/732,831, filed on Sep. 18, 2018.

(51) Int. Cl. A61B 18/02; A61B 18/04

(52) U.S. Cl. CPC A61B 18/02; A61B 2018/00464; A61B 18/04

(57) ABSTRACT

Embodiments described herein are directed to methods of treating visceral fat where the method includes the steps of identifying visceral fat to be treated, inserting a laparoscopic device into the visceral fat to be treated, inserting a treatment instrument into the laparoscopic device such that a distal treatment section of the treatment device is delivered into the visceral fat, cooling the distal treatment section and the visceral fat adjacent to the distal treatment section to a cooling temperature no colder than approximately −20° C., and wanning the distal treatment section and the visceral fat adjacent to the distal treatment section to a temperature greater than the cooling temperature.

### CROSS-REFERENCE TO RELATED APPLICATIONS

This application claims the benefit of U.S. Provisional Application No. 62/732,831, filed Sep. 18, 2018, the entire contents of which are incorporated herein by reference in their entirety for all purposes.

### BACKGROUND

The disclosed and described technology relates generally to the thermal treatment of visceral fat. More specifically, embodiments of the present invention relate to systems, devices and methods for cryolipolysis of visceral fat.

### DESCRIPTION OF THE RELATED TECHNOLOGY

Visceral fat is technically excess intra-abdominal adipose tissue accumulation. In other words, it is known as a “deep” fat that is stored farther beneath the skin than subcutaneous belly fat. Visceral fat is a gel-like fat that wraps around major organs including the liver, pancreas and kidneys. Visceral fat is especially dangerous because the visceral fat cells change the way the body operates.

Carrying excess visceral fat is linked with an increased risk of: coronary heart disease, cancer, stroke, dementia, diabetes, depression, arthritis, obesity, sexual dysfunction and various sleep disorders. (See Neeb Z, Edwards J, Alloosh M, Long X, Mokelke E, Sturek M, “Metabolic Syndrome and Coronary Artery Disease in Ossabaw Compared With Yucatan Swine,” Comparative Medicine (2010) 60:300-15: Després J, Moorjani S, Lupien P, Tremblay A, Nadeau A, Bouchard C, “Regional Distribution of Body Fat, Plasma Lipoproteins, and Cardiovascular Disease,” Arteriosclerosis (1990) 10:497-511; Lemieux I, Pascot A, Prud'homme D, Alméras N, Bogaty P, Nadeau A, Bergeron J, Després J, “Elevated C-Reactive Protein: Another Component of the Atherothrombotic Profile of Abdominal Obesity,” Arteriosclerosis, Thrombosis, and Vascular Biology. (2001) 21:961-67; Pascot A, Lemieux I, Prud'homme D, Tremblay A, Nadeau A, Couillard C, Bergeron J, Lamarche B, Després J, “Reduced HDL Particle Size as an Additional Feature of the Atherogenic Dyslipidemia of Abdominal Obesity,” Journal of Lipid Research (2001) 42:2007-14; Pouliot M. Després J. Nadeau A. Moorjani S, Prud'homme D, Lupien P, Tremblay A, Bouchard C, “Visceral Obesity in Men. Associations With Glucose Tolerance, Plasma Insulin, and Lipoprotein Levels,” Diabetes (1992) 41:826-34; Tchernof A, Lamarche B, Prud'homme D, Nadeau A, Moorjani S, Labrie F, Lupien J, Després J, “The Dense LDL Phenotype. Association With Plasma Lipoprotein Levels, Visceral Obesity, and Hyperinsulinemia in Men,” Diabetes Care (1996) 19:629-37; Ross R, Aru J, Freeman J, Hudson R, Janssen I, “Abdominal Adiposity and Insulin Resistance in Obese Men,” American Journal of Physiology, Endocrinology and Metabolism, (2002) 282:E657-E663; Ross R. Freeman J. Hudson R, Janssen I, “Abdominal Obesity, Muscle Composition. and Insulin Resistance in Premenopausal Women,” The Journal of Clinical Endocrinology and Metabolism, (2002) 87:5044-51; Mertens I, Van der Planken M, Corthouts B, Van Gaal L, “Is Visceral Adipose Tissue a Determinant of Von Willebrand Factor in Overweight and Obese Premenopausal Women?” Metabolism: Clinical and Experimental, (2006) 55:650-55; Brunzell J, Hokanson J, “Dyslipidemia of Central Obesity and Insulin Resistance,” Diabetes Care, (1999) 22 Suppl 3:C10-C13; Nieves D, Cnop M, Retzlaff B, Walden C, Brunzell J, Knopp R, Kahn S, “The Atherogenic Lipoprotein Profile Associated With Obesity and Insulin Resistance is Largely Attributable to Intra-Abdominal Fat,” Diabetes, (2003) 52:172-79; Boyko E, Leonetti D, Bergstrom R, Newell-Morris L, Fujimoto W, “Visceral Adiposity, Fasting Plasma Insulin, and Lipid and Lipoprotein Levels in Japanese Americans,” International Journal of Obesity and Related Metabolic Disorders. (1996) 20: 801-08.)

Visceral fat is considered toxic and is doubly troubling because it's capable of provoking inflammatory pathways and also signals and activates molecules that can interfere with the body's normal hormonal functions. In fact, visceral fat acts like its very own organ because it is capable of having a very large impact on body function as it continuously produces hormones and inflammatory substances.

Storing excess fat around the organs increases the production of pro-inflammatory chemicals/substances called cytokines, which leads to inflammation while at the same time, interferes with hormones that regulate appetite, weight, mood and brain function.

Accordingly, embodiments of the present invention are directed to methods, systems and devices that use non-ablative cold temperatures to treat visceral fat.

### SUMMARY

An aspect of the present invention is directed towards a system for providing alternating cooling and warming cycles. In one embodiment the system includes a controller, a vessel for holding a working fluid, a pressure generator, a cooler, a cooler heat exchanger, a heater, a heater heat exchanger, a check valve, and a treatment instrument. In some embodiments, the treatment instrument includes a distal treatment section, a proximal end, a connecting portion adjacent to the proximal end, and a handle portion disposed between the proximal and distal end. The working fluid can be alcohol ethanol, octafluoropropane, diethyl ether or propylene glycol.

Another aspect of the present invention is directed to a treatment instrument for treating visceral fat. The treatment instrument includes a distal end, a proximal end, a connecting portion adjacent the proximal end, a needle element adjacent the distal end, a distal treatment section adjacent the distal end, and a handle portion disposed between the proximal and distal end. In some embodiments, the treatment device also includes a heating element.

Another aspect of the present invention is directed to a treatment instrument for treating visceral fat, where the treatment instrument comprises a distal end, a proximal end, a connecting portion adjacent the proximal end, a distal treatment section adjacent the distal end and a handle portion disposed between the proximal and distal end. In some embodiments, the distal treatment section comprises a plurality of concentric loops.

A further aspect of the present invention is a method of treating visceral fat. The method comprises the steps of identifying visceral fat to be treated, inserting a laparoscopic device into the visceral fat to be treated, inserting a treatment instrument into the laparoscopic device such that a distal treatment section of the treatment device is delivered into the visceral fat, cooling the distal treatment section and the visceral fat adjacent to the distal treatment section to a cooling temperature no colder than approximately −20° C., and warming the distal treatment section and the visceral fat adjacent to the distal treatment section to a temperature greater than the cooling temperature.

In another embodiment, the invention is directed to a method of treating visceral fat. The method comprises the steps of identifying visceral fat to be treated, inserting a laparoscopic device into a first area of the visceral fat to be treated, inserting a treatment instrument into the laparoscopic device such that a distal treatment section of the treatment device is delivered into the first area of the visceral fat, cooling the distal treatment section and the first area of the visceral fat adjacent to the distal treatment section to a cooling temperature no colder than approximately −20° C., warming the distal treatment section and the first area of the visceral fat adjacent to the distal treatment section to a temperature greater than the cooling temperature, removing the treatment instrument and the distal treatment section of the treatment instrument from the first area of visceral fat, removing the laparoscopic device from the first area of visceral fat, inserting the laparoscopic device into a second area of the visceral fat to be treated, inserting the treatment instrument into the laparoscopic device such that the distal treatment section of the treatment device is delivered into the second area of the visceral fat, cooling the distal treatment section and the second area of visceral fat adjacent to the distal treatment section to a cooling temperature no colder than approximately −20° C. and warming the distal treatment section and the second area of the visceral fat adjacent to the distal treatment section to a temperature greater than the cooling temperature.

Another aspect of the present invention is directed to a method of treating visceral fat where the method comprises the steps of identifying visceral fat to be treated, inserting a treatment instrument into the visceral fat such that a distal treatment section of the treatment device is delivered into a first area of the visceral fat, cooling the distal treatment section and the first area of the visceral fat adjacent to the distal treatment section to a cooling temperature no colder than approximately −20° C., warming the distal treatment section and the first area of the visceral fat adjacent to the distal treatment section to a temperature greater than the cooling temperature, removing the treatment instrument and the distal treatment section of the treatment instrument from the first area of visceral fat, inserting the treatment instrument and the distal treatment section of the treatment instrument into a second area of the visceral fat, cooling the distal treatment section and the second area of visceral fat adjacent to the distal treatment section to a cooling temperature no colder than approximately −20° C., and warming the distal treatment section and the second area of the visceral fat adjacent to the distal treatment section to a temperature greater than the cooling temperature.

In another embodiment, the invention is directed to a method of treating visceral fat. The method comprises the steps of identifying visceral fat to be treated, inserting a treatment instrument into the visceral fat such that a distal treatment section of the treatment device is delivered into a first area of the visceral fat, cooling the distal treatment section and the first area of the visceral fat adjacent to the distal treatment section, warming the distal treatment section and the first area of the visceral fat adjacent to the distal treatment section to a temperature greater than the cooling temperature, removing the treatment instrument and the distal treatment section of the treatment instrument from the first area of visceral fat, inserting the treatment instrument and the distal treatment section of the treatment instrument into a second area of the visceral fat, cooling the distal treatment section and the second area of visceral fat adjacent to the distal treatment section, and warming the distal treatment section and the second area of the visceral fat adjacent to the distal treatment section to a temperature greater than the cooling temperature.

The description, objects and advantages of embodiments of the present invention will become apparent from the detailed description to follow, together with the accompanying drawings.

### BRIEF DESCRIPTION OF THE DRAWINGS

The above-mentioned aspects, as well as other features, aspects, and advantages of the present technology will now be described in connection with various embodiments, with reference to the accompanying drawings. The illustrated embodiments, however, are merely examples and are not intended to be limiting. Throughout the drawings, similar symbols typically identify similar components, unless context dictates otherwise. Note that the relative dimensions of the following figures may not be drawn to scale.

FIG. 1 is thermodynamic phase diagram of a working fluid for use in embodiments of the present invention;

FIG. 2 is a schematic depiction of a system, according to an embodiment of the invention;

FIG. 3 depicts a pressure generator, according to an embodiment of the invention;

FIG. 4 depicts a treatment instrument, according to an embodiment of the invention;

FIG. 5 is a cross-sectional view of a treatment device, according to an embodiment of the invention;

FIG. 6 depicts treatment cycles, according to an embodiment of the invention;

FIG. 7 depicts a treatment instrument, according to an embodiment of the invention;

FIG. 8 depicts a cooling probe having a distal treatment section with a plurality of concentric loops, according to an embodiment of the invention;

FIG. 9A depicts a pattern that the distal treatment section of the probe depicted in FIG. 8 creates in fat tissue when pressed into fat tissue;

FIG. 9B depicts thermal modeling in fat tissue showing the isotherms around the distal treatment section of the probe depicted in FIG. 8;

FIG. 10A is a photo of a loop-shaped distal treatment section of a probe adjacent to visceral fat tissue prior to insertion/pressing into the tissue, according to an embodiment of the invention;

FIG. 10B is a photo of a loop-shaped distal treatment section of a probe inserted into visceral fat tissue, according to an embodiment of the invention;

FIG. 11A is a photo showing the abdominal cavity of an untreated rat;

FIG. 11B is a photo of an abdominal cavity of a rat treated with the device depicted in FIGS. 10A and 10B where the rat received two (2) one (1) minute cooling treatments of −20° C.;

FIG. 12A shows H&E staining of visceral fat tissue obtained from an untreated rat (C);

FIG. 12B shows H&E staining of visceral fat tissue obtained from a rat (T) treated with cryolipolysis:

FIG. 12C is a chart showing the fat cell area (C) calculated using image J software for the untreated rat (FIG. 12A) and the fat cell area (T) calculated using image J software for the treated rat (FIG. 12B); and

FIG. 13 are photos of a TUNNELL assay showing the effects of cooling/freezing on apoptosis in visceral fat.

### DETAILED DESCRIPTION

The disclosed and described technology relates to cryotherapy systems, devices and methods to treat various joint conditions including, and not limited to, arthritis.

### The System and Treatment Instruments:

The system employs an open thermodynamic cycle and uses a cryogen that is a high-density fluid with a freezing temperature below the treatment temperature. The fluid should not be hazardous (such as toxic, explosive, etc.) and must have a high density in order for it to be an efficient thermal agent. The fluid should also have a low viscosity such that it can flow through small channels and small diameter lumens within the treatment device without a significant pressure gradient and/or viscous heating. Sample high density fluids for use in the present system are included in Table 1.

TABLE 1

| | | Freezing | Equilibrium | | Viscosity (μ) |
|---|---|---|---|---|---|
| | Chemical | Temperature | Pressure at | Density | at −25° C. |
| Fluid | Formula | (Celsius) | 25° C. (MPa) | (kg/m³) | (Pa · s) |
| Alcohol Ethanol | C₂H₅OH | −114° | Stable | 890 | 4.0 × 10⁻³ |
| Octafluoropropane | C₃F₈ | −150° | .087 | 1500 | 3.6 × 10⁻⁴ |
| Diethyl Ether | (C₂H₅)₂O | −116° | .100 | 750 | 2.0 × 10⁻⁴ |
| Propylene Glycol | C₃H₈O₂ | −59° | Stable | 1050 | >0.04 |

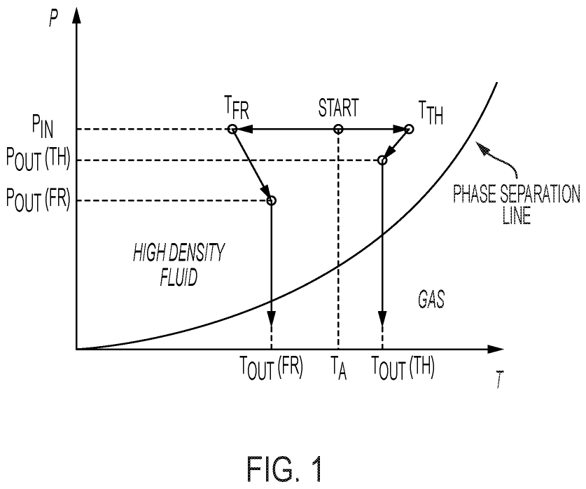

Depicted in FIG. 1 is a simplified thermodynamic phase diagram of the working fluid of the present system. Shown in the figure are the two essential thermodynamic cycles that are required for the embodiments of the treatment methods disclosed and described herein: a Freeze Cycle (FC) and Thaw Cycle (TC). In the figure, the X-axis represents temperature (T) and the Y-axis represents pressure (P).

Initially, the working fluid is maintained at ambient temperature (T_{A}) and an elevated pressure (P_{IN}). Under these conditions, the fluid should be well within its high-density phase. Some examples of a fluid's the high-density phase include the liquid phase and/or supercritical phase, which is where the fluid has the properties of both a liquid and a gas and which typically occurs above the liquid's critical point, i.e., above the liquid's critical temperature and critical pressure.

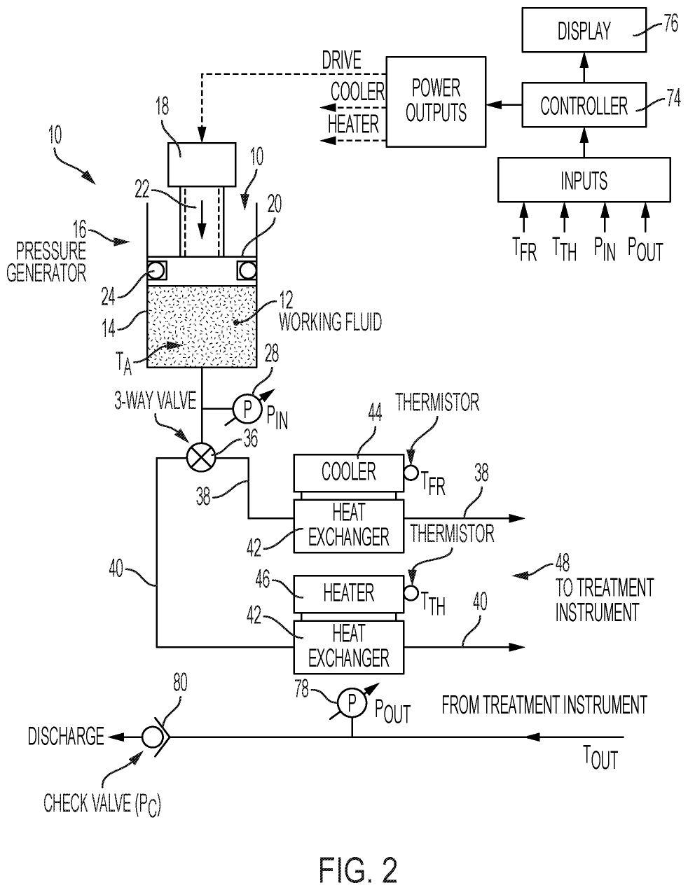

An embodiment of the cryotherapy system 10 is depicted in FIG. 2. Thermal fluid/cryogen 12 is held in a container 14 that is maintained at ambient temperature (T_{A}). The working pressure of the system 10 is maintained by a pressure generator 16. The pressure generator 16 can be any device that is capable of creating/generating the required working pressure for the thermal fluid and maintaining the required pressure for the flow rates required by the system. Non-limiting examples of pressure generators are described herein.

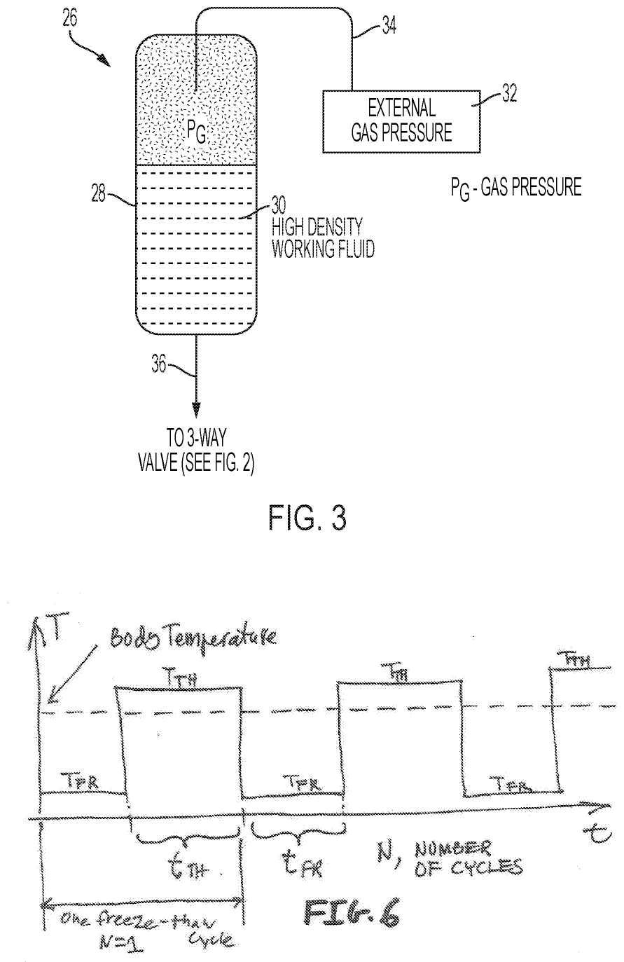

For example, the pressure generator can be a mechanical piston system 18 that includes a piston head 20, a connecting rod/means 22 and at least one compression ring/gasket 24. The piston head 20 and connecting rod 22 can be driven by, for example, an electrical (stepper) motor. The pressures necessary to run the system can also be generated and maintained with the use of a closed volume system that contains the working fluid. As depicted in FIG. 3, this type of closed volume system 26 includes a container/tank 28 to hold the working fluid 30, an external gas pressure source 32 and a pressure line 34 that connects the external gas pressure source 32 to the container/tank 28. In this system, pressurizing the external gas pressure source 32 to a certain gas pressure (PG) also causes the working fluid 30 within the container/tank 28 to pressurize to a certain gas pressure (PG). As will be readily apparent to those skilled in the art, other devices and means can be used to generate the required operating pressures of the system.

Because the system has a dual function of freezing and thawing, a dual fluid flow is included. This is achieved with the use of a three-way valve 36 as depicted in FIG. 2. In use, during a freezing cycle, the three-way valve 36 is actuated to direct working fluid flow along the cooling/freezing flow path 38, and during a thawing cycle, the three-way valve 36 is actuated to direct working fluid flow along the warming/thawing flow path 40. Thus, the three-way valve is used to control the freeze and thaw cycles of the system.

As can be seen in FIG. 2, both the cooling flow path 38 and the warming flow path 40 include heat exchangers 42 to thermally connect the working fluid to both a cooler 44 and a heater 46. The cooler 44 can be any type of cooling device capable of achieving and maintaining the required cooling/freezing temperatures. Example coolers 44 include, and are not limited to: thermoelectric coolers (TEC); cryocoolers such as the Pulse Tube, Stirling. Gifford-McMahon cooler, etc.: Joule-Thomson based coolers that use a gas or liquid supply; evaporative coolers such as refrigerators; and an immersion cooler with a cryogenic liquid. Another example of a cooler that can be used is an evaporative cooler that relies on cold temperatures that are generated by expansion of the high density thermal working fluid used in the present system as a result of the pressure drop in the check valve (discussed below). The heater 46 can be any type of a heating device capable of achieving and maintaining the required heating/thawing temperatures. Example heaters 46 include, and are not limited to: thermoelectric heater.

Also included with the system 10 is a treatment instrument 48 (for example, a needle device) for insertion into skin. The treatment instrument 48 connects to the system through a three-port hermetic connector that connects the cold fluid supply line, warm fluid supply line, and a return fluid line from the system 10 to the treatment instrument 48. In some embodiments, multiple cold fluid supply lines and/or multiple warm fluid supply lines and/or multiple return fluid lines may be used.

Depicted in FIG. 4 is an embodiment of a treatment instrument 48. The treatment instrument 48 includes a small diameter needle 50 at its distal end 52. The needle 50 has a closed distal end with a flow chamber 53 to allow inflowing working fluid to flow through the treatment instrument 48. At its proximal end 54, the treatment instrument 48 includes a connecting portion 56 for thermally connecting to a supply line/hose 58 from the system 10. In some embodiments, the treatment instrument 48 connects to the system with a thermally insulated hose 58 that has at least two insulated lumens to deliver the high density working/thermal fluid to the treatment instrument (one lumen to deliver the cooling fluid and one lumen to deliver the warming fluid) and a lumen for the return flow. As an example, this thermal insulation can be made with Aerogel or it can be vacuum insulation. In order to prevent scarring, the needle 50 can be 27 gauge (or 0.4 mm diameter) and 10 mm length, for example.

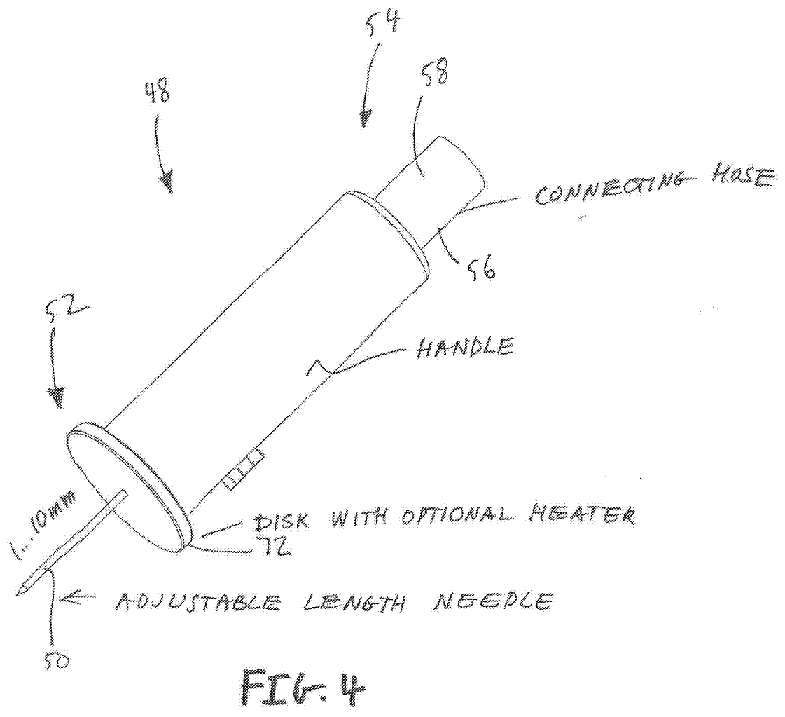

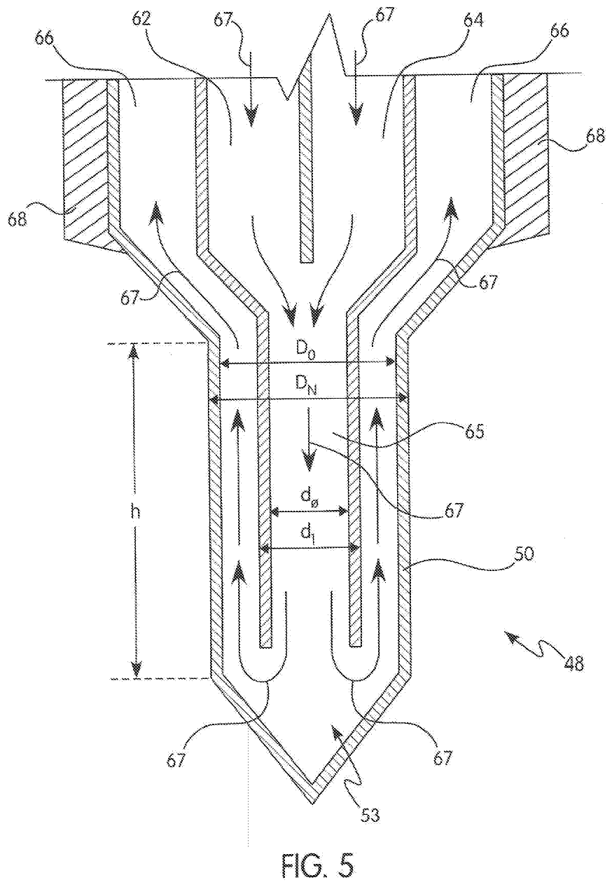

As shown in FIG. 5, the treatment instrument 48 can have a proximal section 60 of a larger diameter in order to house the cooling/freeze channel/lumen 62, the warming/thaw channel/lumen 64 and the fluid return channels/lumens 66. The cooling/freeze channel/lumen 62 and the warming/thaw channel/lumen 64 converge and open into a single delivery channel/lumen 65 for delivery to the flow chamber 53. Thus, as can be seen in FIG. 5, inflowing working fluid 67 (cooling or heating fluid) flows through either of the cooling/freeze channel/lumen 62 or the warming/thaw channel/lumen 64 depending on the treatment cycle (freezing/thawing), into the delivery channel/lumen 65, into to the flow chamber 53 and then exits the flow chamber 53 by flowing into the return channels/lumens 66. In some embodiments, the proximal section 60 includes thermal insulation (vacuum, Aerogel, etc.) 68 to prevent heat loss and avoid moisture condensation and to prevent freezing/heating along this portion of the treatment instrument 48.

In some embodiments, the needle 50 length can range from approximately 1.0 mm to approximately 10.0 mm and extends form a handle portion 70 that, as depicted in FIG. 4, includes a disk-like section 72. The disk-like section 72 is intended to limit the depth that the needle 50 is inserted into the skin tissue under treatment. The insertion depth can be adjusted using a knob, slider, or a dial located on the handle 70. That is, the knob, slider, or dial can be used to either retract the needle 50 within the handle 70 thereby changing the length of the needle 50 that extends from the disk-like section 72. In some embodiments, the knob, slider, or dial can be used move the disk-like section 72 with respect to the needle 50, which also changes the length of the needle 50 that extends from the disk-like section 72. Although in the embodiment depicted in FIG. 4, the disk-like section 72 is shown in the form of a disk, the shape is not limited to a disk but can be any shape as long as it can prevent insertion of the needle 50 past section 72.

In some embodiments, the disk-like section 72 may include a heating element such as, for example, an electrical heater, that is used to prevent freezing of the upper most layer of skin (epidermis) by maintaining the temperature at a safe level, for example, approximately 30° C. to 42° C. The disk-like section 72 may also include a thermal sensor to monitor the temperature of the epidermis temperature in order to control the temperature of the heating element.

Following is a thermodynamic analysis for a needle 50 as depicted in FIG. 5. All of the thermodynamic properties and values used in the below analysis were from the National Institute of Standards and Technology's (NIST) Reference Fluid Thermodynamic and Transport Properties Database (REFPROP).

For the dimensions, assume a standard botox needle, which is 27 gauge. Such a needle can be constructed as follows:

Needle shaft (stainless steel): D_{N}=0.41 mm; D_{O}=0.25 mm

Inner tubing (polyimide): d₁=0.13 mm; d_{ø}=0.10 mm

Annulus space will have its hydraulic diameter (d_{H}) as follows:

d_{H}=D−d₁=0.12 mm OR d_{H}≅d_{ø}

Because the preferred working fluid is octafluoropropane, assume:
TFR=−25° C., which leads to μ=3.6×10⁻⁴ Pa s

Assume laminar flow, for ΔP=P_{IN}−P_{OUT}=100 psi (7×10⁶ Pa)

$$Q = \frac{Δ P \cdot \cdot {{d}_{∅}}^{4}}{128 µL} = \frac{( 7 \times {10}^{5} ) \times ( 3.14 ) \times ( {10}^{- 16} )}{( 128 ) \times ( 3.6 \times {10}^{- 4} ) \times {10}^{- 2}} ≅ 5 \times {10}^{- 7} {m}^{3} / s$$

$$OR$$

$$30 {cm}^{3} / min$$

The corresponding Reynold's Number:

$${R}_{E} = \frac{ρ {vd}_{∅}}{µ} = \frac{( 1.5 ) \times ( 62 \times {10}^{- 4} )}{( 3.6 \times {10}^{- 4} )} ≅ 26$$

Accordingly, the flow is very laminar.

Cooling power=power required to warm up the needle to 0° C.:

$$W - Q \cdot p ( H ( 0 ° C . ) - H ( - 25 ° C . ) ) ; where H is the enthalpy . \\ W = ( 0.5 \frac{{cm}^{3}}{sec} ) \times ( 1.5 \frac{g}{{cm}^{3}} ) \times 24 \frac{J}{s} = 20 \frac{J}{s} = 20 Watts$$

This amount of power is more than adequate to treat visceral fat as discussed herein.

In some embodiments, the system may include multiple treatment instruments, which may be operated independently of one another or which may be operated synchronously. Accordingly, in these embodiments, the system will include multiple connection ports/supply hoses.

As depicted in FIG. 2, the system includes a controller/computer 74 for controlling/managing operation of the system 10. With the controller/computer 74, a user can input the operating parameters for the system 10 such as, for example, freeze temperature (T_{FR}), thaw temperature (T_{TH}), operating pressures (P_{IN}, P_{OUT}), freeze and thaw cycle run times, treatment cycles (freezing/thawing), number of cycles, treatment instrument operation, etc. The controller/computer 74 can be programmed with the operating parameters for different treatment procedures (as discussed below in more detail). Therefore, depending on the treatment procedure that will be performed, a user can simply choose that procedure from a library of procedures that has been programmed into the controller/computer 74 and the system will operate with the operating parameters that are specific for the subject procedure. The controller/computer 74 also allows a user to modify any such pre-programmed operating parameters for a treatment procedure based on personal preference, experience, treatment area conditions, etc. These modifications or changes to the operating parameters can be performed before a procedure commences or during a procedure based on how the procedure is progressing. Also included is a display 76 for displaying information relating to operation of the system 10 and any additional information essential for the treatment being performed.

As depicted in FIG. 6, the treatment cycles of the system 10 consists of alternating periods of freezing (T_{FR}) and thawing (T_{TH}). Each period is characterized by its temperature (T), duration (t), and the number of duty cycles (for example, T_{FR}, T_{TH}, t_{FR}, t_{TH}, N). Thus, one cycle may include one freezing (T_{FR}) period for a set time (t_{FR}) and one thawing (T_{TH}) period for a set time (t_{TH}). The treatment cycles within one treatment can be identical or they may vary in temperature and/or duration.

In order to regulate the temperature for the freezing and thawing cycles, either of the following methods can be used. The freezing temperature (T_{FR}) can be maintained by setting the cooler 44 temperature to some constant value, −25° C., for example, and regulating the fluid flow by changing pressure P_{IN} of the working fluid flowing from the container 14 to the cooler 44. Similarly, the thawing temperature (T_{TH}) can be maintained by setting the heater 46 temperature to some constant value, 38° C., for example, and regulating the fluid flow by changing pressure P_{IN} of the working fluid flowing from the container 14 to the heater 46. Alternatively, the freezing temperature (T_{FR}) can be maintained by setting the pressure P_{IN} of the working fluid flowing from the container 14 to the cooler 44 to a constant pressure and changing the temperature of the cooler 44. Similarly, the thawing temperature (T_{TH}) can be maintained by setting the pressure P_{IN} of the working fluid flowing from the container 14 to the heater 46 to a constant pressure and changing the temperature of the heater 46. Either of the above methods may be used or a combination of the above methods may be used to regulate T_{FR} and T_{TH}.

The system 10 may also include a plurality of sensors such as pressure gauges 78 and thermistors that are used to monitor operation of the system 10 and to control the operating parameters for treatment procedures. Information obtained from these sensors can be displayed on the display 76 so that a user has real-time operating data for the system.

In some embodiments, the system 10 includes a flow meter in the working fluid cooling/freezing flow path 38 in order to measure fluid flow through the system and hence the system's cooling power.

Operation of the system 10 will now be described. The working fluid 12 is first added to container 14, where it is then pressurized by the pressure generator 16 to a predetermined pressure (P_{IN}). Next, for a freeze cycle, the three-way valve 36 is actuated to open the flow path 38 to the cooler 44. The working fluid 12 is then delivered to the heat exchanger 42 for the cooler 44 where the working fluid 12 is cooled to a pre-set treatment freeze temperature (T_{FR}). Once the working fluid 12 is cooled to T_{FR}, the working fluid 12 is delivered the thermally insulated hose 58 to the treatment instrument 48, which is inserted into the target tissue to be cooled/frozen. Because the needle 50 of the treatment device 48 is in thermal contact with the target tissue, heat is removed from the target tissue by the flowing, cooled working fluid, thereby cooling/freezing the target tissue. Within the treatment device 48, the working fluid 12 flows into freeze channel/lumen 62, into the delivery channel/lumen 65, into to the flow chamber 53, then exits the flow chamber 53 by flowing into the return channels/lumens 66 and then exits the treatment device 48 through the return channels/lumens 66. Upon exiting the treatment device 48 through the return channels/lumens 66 and return lumen in the thermally insulated hose 58, the return flow of working fluid, which is now at a higher temperature (T_{OUT}) and lower pressure (P_{OUT}) than it was before flowing through the treatment instrument 48, is delivered back to the console, which houses many of the system's components, and discharged to the atmosphere via a check valve 80 that is pre-set to a certain release pressure (P_{C}). Using a check valve with a pre-set release pressure is required in order to maintain the working fluid in its high-density state throughout the freeze cycle. The pre-set release pressure (P_{C}) of the check valve 80 is determined by the choice of working fluid that is used in the system 10. That is, different pressures are required to be maintained for different working fluids in order to maintain the working fluids in their high-density state. It is important to note that from the time the working fluid 12 is pressurized and leaves the container 14 until the time it is discharged to the atmosphere through the check valve 80, the working fluid 12 always remains in its high density state as can be seen in FIG. 1. The flow rate of the working fluid 12 through the system 10 is determined by the difference between its initial pressure P_{IN} and the pressure P_{OUT}

The thaw cycle is similar to the freeze cycle except that the flow path of the working fluid 12 in the system 10 is different. For the thaw cycle, the three-way valve 36 is actuated to open the flow path 38 to the heater 46. The working fluid 12 is then delivered to the heat exchanger 42 for the heater 46 where the working fluid 12 is heated to a pre-set treatment thaw temperature (T_{TH}). Once the working fluid 12 is heated to T_{TH}, the working fluid 12 is delivered the thermally insulated hose 58 to the treatment instrument 48, which is inserted into the target tissue to be heated/thawed. Operation of the system 10 for all other aspects is similar to that of the freeze cycle. Again, as shown in FIG. 1, the working fluid remains in its high-density state at all times during the thaw cycle while flowing in the system 10 until the check valve 80.

In another embodiment, the system can be a closed loop system. As used herein, “closed loop” means that instead of venting working fluid through a check valve to the atmosphere after it flows through the treatment instrument for either freezing or thawing, the working fluid is instead returned to the holding container for re-use by the system. This can be achieved by means of an external pump.

It is important to note that unlike prior systems (argon-based systems, for example), the cooling/freezing and warming/thawing effect in the present system does not occur at the treatment device. Instead, cooling and heating of the working fluid 12 are achieved using a dedicated cooler or heater prior to the working fluid entering the treatment device.

### Treatment Methods

Procedures and methods to treat visceral fat using the disclosed and described systems and treatment devices will now be described. Embodiments of the present invention expose visceral fat to non-ablative cooling temperatures warmer than approximately −20° C. in order to induce fat cell apoptosis. The temperatures used induce fat cell apoptosis but do not have any deleterious effects on surrounding tissue and organs.

In some embodiments, the treatment device is inserted into the body and into the visceral fat using laparoscopic devices and methods. As is known by those of skill in the art, an incision can be made in the skin and the laparoscopic instrument, which can include a camera, is inserted through the skin and navigated to the visceral fat to be treated. As will be understood by those of skill in the art, other delivery devices may be used in place of lapascopic instruments to deliver the treatment device to the visceral fat.

Once the laparoscopic instrument is in place at the treatment site, the treatment device, which as depicted in FIG. 7, can be a long probe 500 with a cooling distal treatment section 505, is inserted into the laparoscopic instrument and delivered to the visceral fat treatment site. Once in place, the cooling distal treatment section 505 is cooled to no lower than −20° C. In some embodiments, temperatures greater than (warmer) than −20° C. may be used to treat the visceral fat. In some embodiments, the probe 500 can be rigid or it can be flexible. As depicted in FIG. 7, the probe 500 includes a blunt tip 510 in order to prevent the probe 500 from puncturing tissue adjacent to the treatment site such as, for example, body organs, etc.

The length “L” of the distal treatment section 505 can vary from patient to patient and can be based on the target tissue being treated. Probes 500 can be manufactured to have different length distal treatment sections 505 or the length of the distal treatment section can be controlled by controlling the length of the distal treatment section that is exposed from the distal end of the laparoscope or other device used to deliver the probe 500 to the target tissue.

Treating visceral fat requires the distal treatment section 505 of the probe 500 to be maneuvered to different locations within the treatment are, which contains the visceral fat. Thus, in some embodiments, the probe 500 includes a warming function, which can be achieved in accordance with the embodiments disclosed and described herein. During treatments, after cooling the distal treatment section 505, the distal treatment section 505 can be warmed up to “unstick” the distal treatment section 505 from the location within visceral fat being treated. The probe 500 and hence the distal treatment section 505 can then be moved to another location within the visceral fat. This allows the procedure to be sped up as the distal treatment section 505 can be actively warmed to release it from tissue instead of waiting for the body tissue to naturally warm-up the distal treatment section 505, which can take time. In some embodiments, the treatment can include alternating cooling and warming cycles within the same location in the visceral fat.

In another embodiment, the treatment method includes identifying visceral fat to be treated. Once identified, a laparoscopic device or other delivery device is inserted into a first area of the visceral fat to be treated. After the delivery device is inserted at the desired location, the treatment instrument 500 is inserted into the delivery device such that the distal treatment section 505 of the treatment device 500 is delivered into the first area of the visceral fat. When in place, the distal treatment section 505 and the first area of the visceral fat adjacent to the distal treatment section 505 are cooled by circulating a working fluid through the distal treatment section 505 to a cooling temperature no colder than approximately −20° C. After a desired cooling time/period, which, in some embodiments can be approximately 10 minutes, the distal treatment section 505 and the first area of the visceral fat adjacent to the distal treatment section 505 are warmed to a temperature greater than the cooling temperature. In some embodiments, additional cooling and warming cycles are performed at the first area of the visceral fat. When the desired number of cooling and warming cycles are performed, the treatment instrument 500 and the distal treatment section 505 of the treatment instrument 500 are removed from the first area of visceral fat. In some embodiments, the treatment includes inserting the treatment instrument 500 and the distal treatment section 505 into a second area of the visceral fat and cooling the distal treatment section 505 and the second area of visceral fat adjacent to the distal treatment section 505 to a cooling temperature no colder than approximately −20° C. After the desired cooling time/period, the distal treatment section 505 and the second area of the visceral fat adjacent to the distal treatment section 505 are warmed to a temperature greater than the cooling temperature. In some embodiments, additional cooling and warming cycles are performed at the second area of the visceral fat. When the desired number of cooling and warming cycles are performed, the treatment instrument 500 and the distal treatment section 505 of the treatment instrument 500 are removed from the second area of visceral fat. In some embodiments, 3 and/or 4 and/or 5 and/or 6 and/or any additional number of areas of the visceral fat are cooled and warmed as disclosed and described herein. In some embodiments, instead of actively warming the areas of the visceral fat after the cooling cycle, the areas of the visceral fat are allowed to naturally warm up as a result of body temperature and not through active warming from the distal treatment section 505.

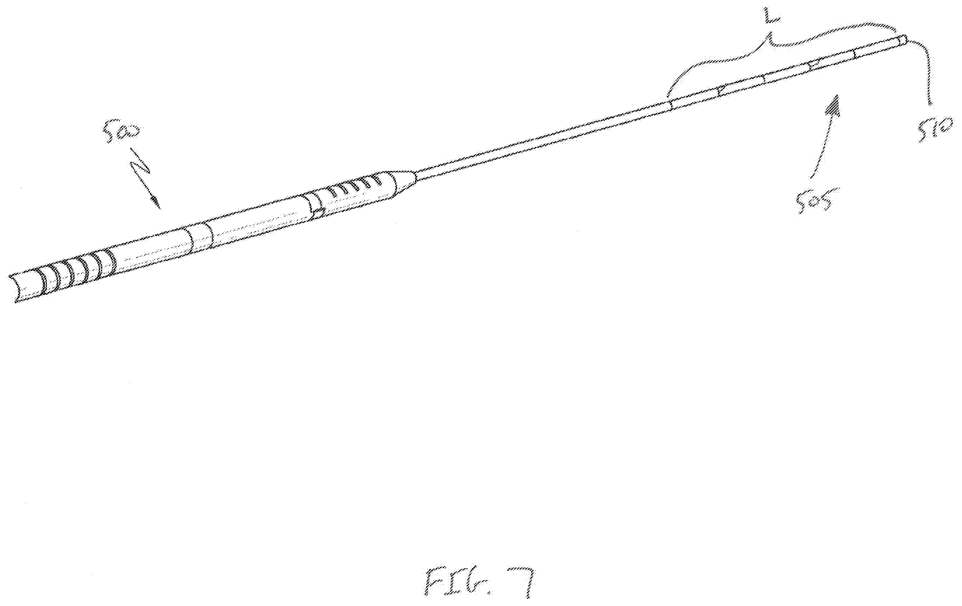

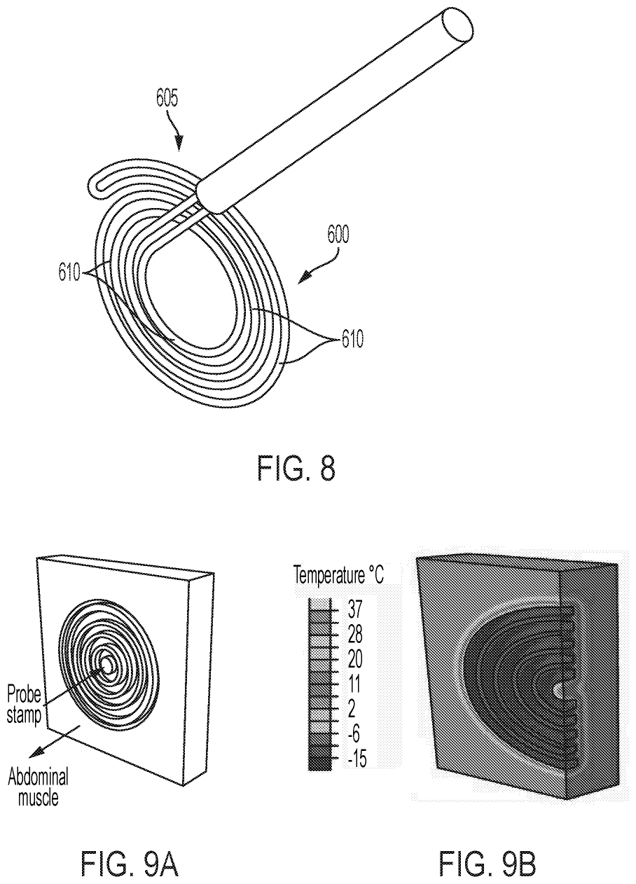

Another embodiment of the invention is depicted in FIG. 8. As can be seen in FIG. 8, the distal treatment section 600 includes a spiral or plurality of concentric loops configuration 605. In some embodiments, the distal treatment section 600 can be constructed from a super elastic alloy such as, for example, nitinol (NiTi) alloy. Cooling/freezing can be achieved by flowing the cooling fluid through the distal treatment section thereby, resulting in cooling of the distal treatment section to a desired temperature. In some embodiments, the distal treatment section 600 can be retrieved inside the guiding sheath in order to help control full or partial insertion into the tissue to be treated.

The distal treatment section 600 of this embodiment can be deployed through a guiding sheath laparoscopically. In this embodiment, the dimensions of the spiral or plurality of concentric loops configuration 605 is approximately 10.5 cm in diameter and forms four (4) loops 610. As will be understood by those of skill in the art, the loop configuration 605 can be designed to have different diameters with a different number of loops 610 depending on the size of fat tissue to be treated. The distance between the individual loops 610 was constant at 0.5 cm with a penetration depth into the tissue (P) at 2, 4 and 6 mm. Simplified anatomy was used for the location of the visceral fat and the cooling probe was analyzed to −15° C. while pressing the distal treatment section into the visceral fat tissue (see FIG. 9A). A numerical simulation of the temperature distribution after sixty (60) seconds of probe cooling to −15° C. is shown in FIG. 9B. Analyzing different penetrations of the loop configuration 605 into the visceral fat tissue, we found that with this configuration, the probe can cool over 40 cm³ of fat to less than about −10° C. in three (3) minutes. Because the device can include both heating/cooling cycles, this cooling can be repeated many times covering a large volume of the total visceral fat.

Experimental Results

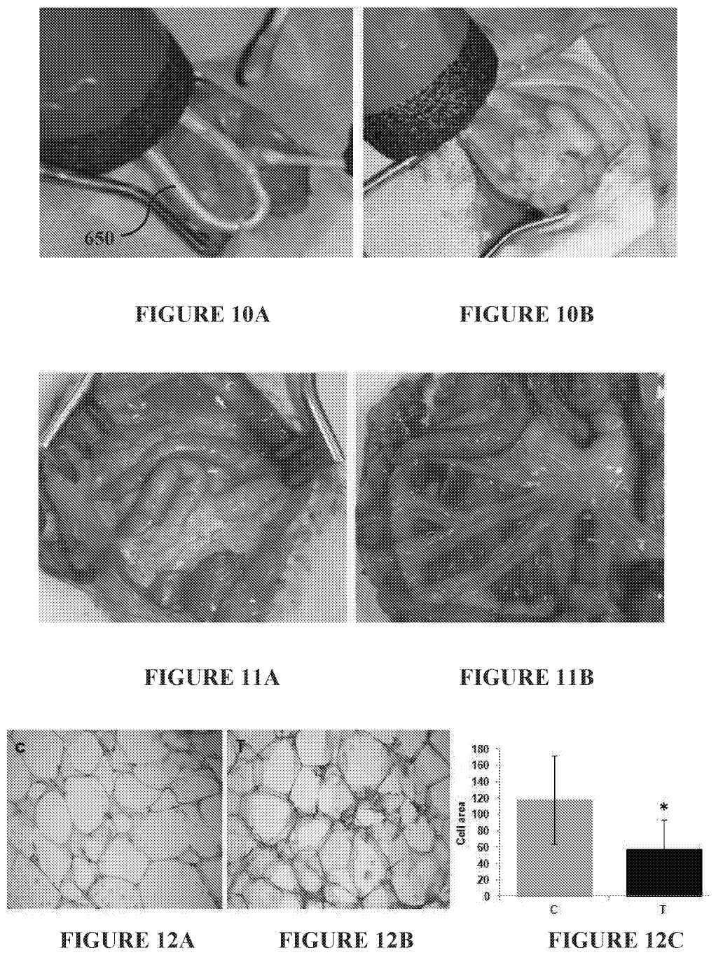

To show that the embodiments of the present invention can cool/freeze visceral fat and promote fat cell loss, a prototype was built with a distal treatment section having one loop 650 (see FIGS. 10A and 10B) capable of delivering cooling/freezing temperatures to the fat tissue. Three Wistar rats were used in this experiment where one rat was the control and was not treated and the other two (2) rates were treated by cooling the distal treatment section to two (2) different temperatures (−5° C. and −20° C.). Both cooling/freezing times were for one (1) minute. Temperature during each cooling/freezing cycle was monitored using a thermocouple mounted on the distal treatment section. The distal treatment section was pushed/inserted into the fat tissue as depicted in FIG. 10B without concern of preventing contact with other surrounding internal organs to show safety even if other organs are in close proximity to the distal treatment section. After the cooling/freezing treatments were performed on the two (2) rats, the rats were allowed to recover for five (5) days post procedure. Both treated rats survived the procedure and no damage to surrounding tissue was macroscopically observed (see FIG. 11B). At day 5, all three (3) rats were euthanized and the abdominal visceral fat tissue was collected for from each for analysis.

Visceral fat tissue was fixed (4% paraformaldehyde) and cryo-processed for hematoxylin-eosin (H&E) staining (see FIGS. 12A and 12B). Fat cells from the control (untreated) rat (C) depicted in FIG. 12A were larger in size and the tissue had no signs of stress or inflammation. In contrast, the fat cells for the rats (T) treated with cryolipolysis (the visceral fat cells underwent a cooling/freeze cycle) depicted in FIG. 12B appear smaller in size and overall the tissue looked stressed and inflamed. The fat cell area was calculated using image J software and confirmed that the fat cell area was smaller after cryolipolysis mean t SD (*p<0.01 T vs. C; n=50 cells).

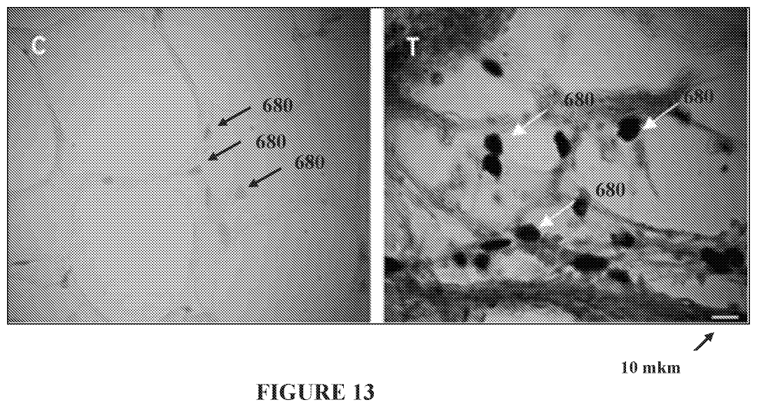

Additionally, the inventors measured the effect of cooling/freezing on apoptosis in visceral fat using the TUNNEL assay (see FIG. 13). With this assay, cells containing fragmented nuclear chromatin characteristic of apoptosis will exhibit brown nuclear staining that may be very dark after labeling. The results of the experiments described herein show increased apoptosis five (5) days after cryolipolysis. Furthermore, Methyl Green was used to counterstain the cell nuclei. This counterstaining revealed an increase in cell numbers in the treated group suggestive of inflammatory cell infiltration into the tissue.

As depicted in FIG. 13, the TUNNEL assay shows increased apoptosis five (5) days after cryolipolysis in the treated rats (T), where arrows 675 point to apoptotic cells. Methy Green was used to counterstain the cell nuclei. As identified by arrows 680 in FIG. 13. Bar in T is 10 μm.

Because cooling fat to temperatures of approximately 10° C. of colder induces fat cell apoptosis, treating visceral fat in accordance with the disclosed and described embodiments, can significantly reduce the mass of the visceral fat tissue thereby reducing and even eliminating the adverse effects caused by visceral fat as discussed herein.

The foregoing disclosure provides for embodiments of systems, devices and methods for treating joint conditions such as, for example, arthritis, etc. While several components, techniques and aspects have been described with a certain degree of particularity, it is manifest that many changes can be made in the specific designs, constructions and methodology herein above described without departing from the spirit and scope of this disclosure.

It is to be understood that the embodiments of the invention described herein are not limited to particular variations set forth herein as various changes or modifications may be made to the embodiments of the invention described and equivalents may be substituted without departing from the spirit and scope of the embodiments of the invention. As will be apparent to those of skill in the art upon reading this disclosure, each of the individual embodiments described and illustrated herein has discrete components and features that may be readily separated from or combined with the features of any of the other several embodiments without departing from the scope or spirit of the embodiments of the present invention. In addition, many modifications may be made to adapt a particular situation, material, composition of matter, process, process act(s) or step(s) to the objective(s), spirit or scope of the embodiments of the present invention. All such modifications are intended to be within the scope of the claims made herein.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain embodiments include or do not include, certain features, elements, and/or steps. Thus, such conditional language is not generally intended to imply that features, elements, and/or steps are in any way required for one or more embodiments.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It will be understood that when an element is referred to as being “connected” or “coupled” to another element, it can be directly connected or coupled to the other element or intervening elements may be present. In contrast, if an element is referred to as being “directly connected” or “directly coupled” to another element, there are no intervening elements present.

It will be understood that, although the terms first, second, etc. may be used herein to describe various elements, these elements should not be limited by these terms. These terms are only used to distinguish one element from another. Thus, a first element could be termed a second element without departing from the teachings of the present invention.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. Additionally, numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth.

Some embodiments have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

While a number of embodiments and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A system for providing alternating cooling and warming cycles, the system comprising:
a controller;
a vessel for holding a working fluid;
a pressure generator;
a cooler;
a cooler heat exchanger,
a heater;
a heater heat exchanger;
a check valve; and
a treatment instrument comprising:
a distal treatment section;
a proximal end;
a connecting portion adjacent the proximal end; and
a handle portion disposed between the proximal and distal end.

2. The system of claim 1, wherein the pressure generator comprises a piston for raising a pressure of the system.

3. The system of claim 2, wherein the piston is driven by a stepper motor.

4. The system of claim 1, wherein the pressure generator comprises an external gas pressure source.

5. The system of claim 1, further comprising a three-way valve located between the vessel and the cooler and the heater.

6. The system of claim 1, further comprising a working fluid.

7. The system of claim 6, wherein the working fluid is selected from the group consisting of ethanol, octafluoropropane, diethyl ether and propylene glycol.

8. A treatment instrument for treating visceral fat, the treatment instrument comprising:
a distal end;
a proximal end;
a connecting portion adjacent the proximal end;
a distal treatment section adjacent the distal end; and
a handle portion disposed between the proximal and distal end.

9. The treatment device of claim 8, further comprising a heating element.

10. The treatment device of claim 9, wherein the distal treatment section further comprises a needle element and the heating element is located adjacent to the needle element.

11. The treatment device of claim 8, wherein the distal treatment section comprises a plurality of concentric loops.

12. A method for treating visceral fat comprising the steps of:
identifying visceral fat to be treated;
inserting a laparoscopic device into the visceral fat to be treated;
inserting a treatment instrument into the laparoscopic device such that a distal treatment section of the treatment instrument is inserted into the visceral fat to be treated;
performing a cooling cycle comprising cooling the distal treatment section and the visceral fat to be treated adjacent to the distal treatment section to a cooling temperature of no less than approximately −20° C.; and
performing a warming cycle comprising warming the distal treatment section and the visceral fat to be treated adjacent to the distal treatment section to a temperature greater than the cooling temperature.

13. The method of claim 12, wherein a plurality of cooling cycles and warming cycles are performed in the visceral fat to be treated.

14. A method for treating visceral fat comprising the steps of:
identifying visceral fat to be treated;
inserting a laparoscopic device into a first area of the visceral fat to be treated;
inserting a treatment instrument into the laparoscopic device such that a distal treatment section of the treatment instrument is inserted into the first area of the visceral fat to be treated;
performing a cooling cycle comprising cooling the distal treatment section and the first area of the visceral fat to be treated adjacent to the distal treatment section to a cooling temperature of no less than approximately −20° C.;
performing a warming cycle comprising warming the distal treatment section and the first area of the visceral fat to be treated adjacent to the distal treatment section to a temperature greater than the cooling temperature;
removing the distal treatment section of the treatment instrument from the first area of visceral fat to be treated;
removing the treatment instrument from the laparoscopic device;
removing the laparoscopic device from the first area of visceral fat to be treated;
inserting the laparoscopic device into a second area of the visceral fat to be treated;
inserting the treatment instrument into the laparoscopic device such that the distal treatment section of the treatment instrument is inserted into the second area of the visceral fat to be treated;
performing a cooling cycle comprising cooling the distal treatment section and the second area of visceral fat to be treated adjacent to the distal treatment section to a cooling temperature of no less than approximately −20° C.; and
performing a warming cycle comprising warming the distal treatment section and the second area of the visceral fat to be treated adjacent to the distal treatment section to a temperature greater than the cooling temperature.

15. The method of claim 14, wherein a plurality of cooling cycles and warming cycles are performed at the first area of visceral fat to be treated.

16. The method of claim 15, wherein a plurality of cooling cycles and warming cycles are performed at the second area of visceral fat to be treated.

17. A method for treating visceral fat comprising the steps of:
identifying visceral fat to be treated;
inserting a treatment instrument into the visceral fat to be treated such that a distal treatment section of the treatment instrument is inserted into a first area of the visceral fat to be treated;
cooling the distal treatment section and the first area of the visceral fat to be treated adjacent to the distal treatment section to a cooling temperature of no less than approximately −20° C.;
warming the distal treatment section and the first area of the visceral fat to be treated adjacent to the distal treatment section to a temperature greater than the cooling temperature;
removing the treatment instrument from the visceral fat to be treated such that the distal treatment section of the treatment instrument is removed from the first area of visceral fat to be treated;
inserting the treatment instrument into the visceral fat to be treated such that the distal treatment section of the treatment instrument is inserted into a second area of the visceral fat to be treated;
cooling the distal treatment section and the second area of visceral fat to be treated adjacent to the distal treatment section to a cooling temperature of no less than approximately −20° C.; and
warming the distal treatment section and the second area of the visceral fat to be treated adjacent to the distal treatment section to a temperature greater than the cooling temperature.

18. A method for treating visceral fat comprising the steps of:
identifying visceral fat to be treated;
inserting a treatment instrument into the visceral fat to be treated such that a distal treatment section of the treatment instrument is inserted into a first area of the visceral fat to be treated;
cooling the distal treatment section and the first area of the visceral fat to be treated adjacent to the distal treatment section to a cooling temperature;
warming the distal treatment section and the first area of the visceral fat to be treated adjacent to the distal treatment section to a temperature greater than the cooling temperature;
removing the treatment instrument from the visceral fat to be treated such that the distal treatment section of the treatment instrument is removed from the first area of the visceral fat to be treated;
inserting the treatment instrument into the visceral fat to be treated such that the distal treatment section of the treatment instrument is inserted into a second area of the visceral fat to be treated;
cooling the distal treatment section and the second area of visceral fat to be treated adjacent to the distal treatment section to a cooling temperature; and
warming the distal treatment section and the second area of the visceral fat to be treated adjacent to the distal treatment section to a temperature greater than the cooling temperature.
